(19) United States
(12) Patent Application Publication
(10) Pub. No.: US 2025/0027854 A1
(43) Pub. Date: Jan. 23, 2025

(54) WATER QUALITY MEASUREMENT DEVICE

(71) Applicant: KURITA WATER INDUSTRIES LTD., Tokyo (JP)

(72) Inventors: Jun KANEKO, Tokyo (JP); Masayuki MIWA, Tokyo (JP); Minoru ARAI, Tokyo (JP)

(73) Assignee: KURITA WATER INDUSTRIES LTD., Tokyo (JP)

(21) Appl. No.: 18/714,583

(22) PCT Filed: Oct. 20, 2022
(86) PCT No.: PCT/JP2022/039050

§ 371 Date: May 30, 2024

(30) Foreign Application Priority Data
Dec. 13, 2021 (JP) 2021-201385

(51) Int. Cl. G01N 1/38; G01N 33/18

(52) U.S. Cl. CPC G01N 1/38; G01N 33/182; G01N 2001/383

(57) ABSTRACT

Provided is a water quality measurement device, comprising: a dilution device, preparing sample water including raw water and dilution water; and a residual chlorine concentration measurement device, measuring a residual chlorine concentration of the sample water, wherein the dilution device comprises: a container in which the sample water is accommodated; a first liquid level detection portion, detecting a first liquid level in the container; a second liquid level detection portion, detecting a second liquid level in the container; a raw water injection portion, injecting the raw water into the container until the first liquid level detection portion detects the first liquid level; and a dilution water injection portion, diluting the raw water by injecting the dilution water into the container until the second liquid level detection portion detects the second liquid level.

### TECHNICAL FIELD

The present invention relates to a water quality measurement device. More specifically, the present invention relates to a water quality measurement device including a dilution device and a residual chlorine concentration measurement device.

### BACKGROUND ART

Batch type automatic continuous residual chlorine concentration measurement devices that measures the residual chlorine concentration in water by absorptiometric method using N,N-diethyl-1,4-phenylenediamine reagent (DPD reagent) are known as a technology to automatically measure the residual chlorine concentration in water. The residual chlorine concentration measurable by the device is 8 mg/L or less. Thus, the device is not suitable for measuring a high concentration region of more than 8 mg/L.

The following Patent Document 1 describes a measurement device of the residual chlorine concentration, which may avoid outputting erroneous measured values when the residual chlorine concentration of the water to be measured exceeds the measurable range. The measurement device described in Patent Document 1 may determine whether the residual chlorine concentration is within the measurable range, but does not have a function to measure the residual chlorine concentration when it is not within the measurable range.

### PRIOR ART DOCUMENT

### [Patent Document]

Patent Document 1: Japanese Patent Application Laid-Open (JP-A) No. 2007-93398

### SUMMARY

### Issues to be Solved by the Present Invention

When residual chlorine concentration is high, measurement of the residual chlorine concentration is generally performed by manual analysis. In order to reduce the workload of manual analysis, there is a need for a technology that may automatically quantify high concentration residual chlorine.

Thus, the main purpose of the present invention is to provide a device that may quantify a high concentration residual chlorine.

### Means for Solving the Issues

That is, the present invention provides a water quality measurement device comprising:
a dilution device, preparing sample water including raw water and dilution water; and
a residual chlorine concentration measurement device, measuring a residual chlorine concentration of the sample water,
wherein the dilution device comprises:
a container in which the sample water is accommodated;
a first liquid level detection portion, detecting a first liquid level in the container;
a second liquid level detection portion, detecting a second liquid level in the container;
a raw water injection portion, injecting the raw water into the container until the first liquid level detection portion detects the first liquid level; and
a dilution water injection portion, diluting the raw water by injecting the dilution water into the container until the second liquid level detection portion detects the second liquid level.

The dilution device comprises:
a discharge portion, discharging the sample water accommodated in the container until the first liquid level detection portion detects the first liquid level, and
after the discharge portion discharges the sample water, the dilution water injection portion may re-dilute the raw water by injecting the dilution water into the container until the second liquid level detection portion detects the second liquid level.

The raw water may be re-diluted for two or more times in the dilution device.

The dilution device may include a circulation portion that circulates the sample water within the container.

The residual chlorine concentration measurement device may measure the residual chlorine concentration of the sample water based on an absorbance of the sample water colored by a reagent.

The reagent may be an N,N-diethyl-1,4-phenylenediamine reagent.

The residual chlorine concentration of the raw water may be more than 8 mg/L.

The raw water may be washing water for cleaning in place.

### Effects of the Invention

The present invention provides a device that may quantify a high concentration of residual chlorine. It is noted that the effect of the present invention is not limited to that described herein and may be any of the effect described in this description.

### BRIEF DESCRIPTION OF THE DRAWINGS

FIG. 1 is schematic view showing an example of the water quality measurement device.

FIG. 2 is a schematic view showing the container and the circulation portion.

FIG. 3 is a schematic view showing the sample water storage portion.

### DESCRIPTION OF THE EMBODIMENTS

Preferable embodiments for carrying out the present invention are described below. It is noted that the described embodiments are just typical embodiments of the present invention and the scope of the present invention should not be limited thereto.

### 1. Configuration and Operation of Water Quality Measurement Device

The water quality measurement device according to an embodiment of the present invention includes: a dilution device, preparing sample water including raw water and dilution water; and a residual chlorine concentration measurement device, measuring a residual chlorine concentration of the sample water. The object of measurement of the residual chlorine concentration measurement device is not the raw water itself, but sample water prepared by diluting the raw water. Thus, even when the residual chlorine concentration of the raw water exceeds the concentration range measurable by the residual chlorine concentration measurement device, the residual chlorine concentration of the raw water may be quantified based on the residual chlorine concentration of the sample water.

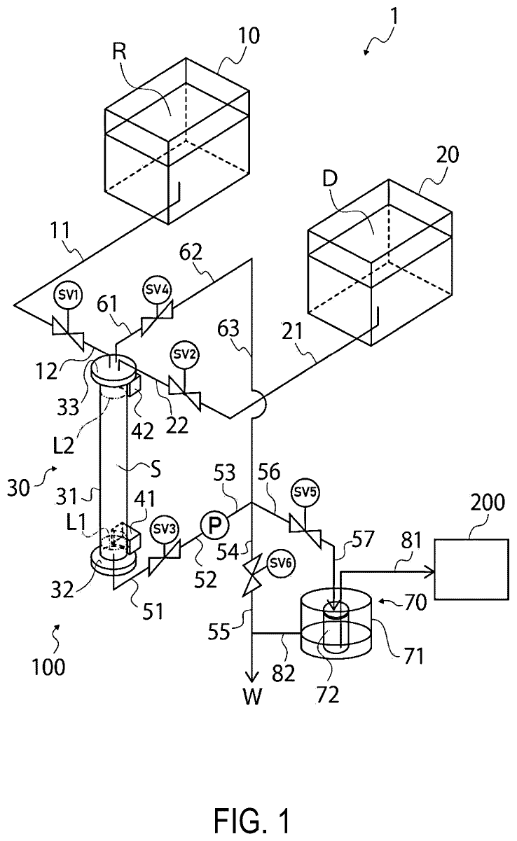

With reference to FIG. 1, the water quality measurement device 1 of this embodiment will be described. FIG. 1 is schematic view showing an example of the water quality measurement device 1. The water quality measurement device 1 includes a dilution device 100 and a residual chlorine concentration measurement device 200. As shown in FIG. 1, the water quality measurement device 1 preferably includes a sample water storage portion 70.

The dilution device 100 prepares sample water S by diluting raw water R and re-diluting the raw water R as necessary, and transfers the sample water S to the sample water storage portion 70. The sample water S in the sample water storage portion 70 is transferred to the residual chlorine concentration measurement device 200. The residual chlorine concentration measurement device 200 performs a measurement on the residual chlorine concentration of the sample water S. In the water quality measurement device 1, a series of processes including the preparation of the sample water S and the measurement of the residual chlorine concentration may be performed once or twice or more. By performing this series of processes two or more times, the water quality measurement device 1 may continuously perform quantification on the residual chlorine concentration of the raw water R over time.

The configurations and operations of the dilution device 100, the sample water storage portion 70, and the residual chlorine concentration measurement device 200 will be described below in order.

### 1-1. Dilution Device

The dilution device 100 will be described with reference to FIG. 1. The dilution device 100 prepares the sample water S consisting of the raw water R and the dilution water D. The sample water S is an example, and the sample water prepared in the dilution device may, for example, include components other than raw water and dilution water. As shown in FIG. 1, the raw water R may be, for example, accommodated in a raw water tank 10. The dilution water D may be, for example, accommodated in a sample water tank 20.

Although the upper limit of the residual chlorine concentration of the raw water R varies depending on the dilution performance of the dilution device 100 and the measurement performance of the residual chlorine concentration measurement device 200, it may be, for example, 8000 mg/L or less. The lower limit of the residual chlorine concentration of the raw water R is, for example, 8 mg/L or more, more than 8 mg/L, 50 mg/or more, 100 mg/L or more, 200 mg/L or more, or 500 mg/L or more.

The dilution device 100 includes a container 30 in which the sample water S is accommodated. The container 30 includes, for example, a cylindrical container body 31, a bottom surface portion 32 provided at one end of the container body 31, and a top surface portion 32 provided at the other end of the container body 31. The container body 31 is the main body part in which the sample water S is accommodated. The bottom surface portion 32 is located on the bottom surface of the container 30. For example, pipes, which will be described later, may be connected to the bottom surface portion 32. The top surface portion 33 is located on the top surface of the container 30. For example, pipes, which will be described later, may be connected to the top surface portion 33.

The shape of the container body 31 may be cylindrical as described above, but is not limited thereto, and may be any shape appropriately selected by those skilled in the art. Examples of shapes other than a cylindrical shape include a rectangular tube shape, a column shape, and a box shape. The size of the container body 31 may be appropriately set by a person skilled in the art in accordance with the amount of the sample water S (the amount of the raw water R and the dilution water D) to be accommodated. As an example, when the container body 31 is cylindrical, the outer diameter may be 20 mm to 30 mm, the inner diameter may be 15 mm to 25 mm, and the height may be 300 mm to 400 mm, but is not limited thereto. The material of the container body 31 is preferably one that has high resistance to chlorine, and may be, for example, polyacrylic resin.

The dilution device 100 includes a first liquid level detection portion 41 that detects a first liquid level L1 in the container 30 and a second liquid level detection portion 42 that detects a second liquid level L2 in the container 30. In the container 30, the second liquid level L2 is located above the first liquid level L1. It is noted that, in this description, the upper part of the container 30 means the upper part when the top surface portion 33 side is the upper side and the bottom surface portion 32 side is the lower side.

The first liquid level detection portion 41 and the second liquid level detection portion 42 may be configured by known liquid level detection means. Examples of the liquid level detection means include electrostatic capacity type, float type, electrode type, optical type, ultrasonic type, guide pulse type, and pressure type liquid level sensors.

The first liquid level detection portion 41 is configured in the container 30 at a position where the first liquid level L1 may be detected. The second liquid level detection portion 42 is configured in the container 30 at a position where the second liquid level L2 may be detected. The positions where the first liquid level detection portion 41 and the second liquid level detection portion 42 are configured may be determined as appropriate in accordance with the characteristics of the liquid level detection means used as the first liquid level detection portion 41 and the second liquid level detection portion 42.

The dilution device 100 shown in FIG. 1 includes two electrostatic capacity type liquid level sensors on the outside of the container body 31 as the first liquid level detection portion 41 and the second liquid level detection portion 42. Specifically, the two sensor bodies of the electrostatic capacity type liquid level sensors are configured at positions corresponding to the first liquid level L1 and the second liquid level L2, respectively.

The dilution device 100 includes a raw water injection portion that injects the raw water R into the container 30 until the first liquid level detection portion 41 detects the first liquid level L1. The raw water injection portion may include, for example, a pipe 11, a solenoid valve SV1, and a pipe 12 that connect the raw water tank 10 and the container 30. The pipe 12 may be, for example, connected to the top surface portion 33 of the container 30. The opening or closing of the solenoid valve SV1 allows or disallows the raw water R in the raw water tank 10 to be injected into the container 30. Specifically, by opening the solenoid valve SV1 until the first liquid level detection portion 41 detects the first liquid level L1, the raw water R in the raw water tank 10 is injected into the container 30 through the pipes 11 and 12. After that, when the first liquid level detection portion 41 detects the first liquid level L1, the injection of the raw water R into the container 30 is stopped by closing the solenoid valve SV1. The opening or closing of the solenoid valve SV1 may be controlled, for example, by a control device (not shown) electrically connected to the solenoid valve SV1. It is noted that in this specification, the solenoid valve is an example of a valve, and a valve other than the solenoid valve may also be selected.

A predetermined amount of raw water R may be injected into the container 30 by operating the raw water injection portion as described above. The amount of raw water R to be injected is increased or decreased by changing the position of the first liquid level L1 detected by the first liquid level detection portion 41 upward or downward.

The dilution device 100 includes a dilution water injection portion that dilutes the raw water R by injecting the dilution water D into the container 30 until the second liquid level detection portion 42 detects the second liquid level L2. The dilution water injection portion may include, for example, a pipe 21, a solenoid valve SV2, and a pipe 22 that connect the dilution water tank 20 and the container 30. The pipe 22 may be, for example, connected to the top surface portion 33 of the container 30. The opening or closing of the solenoid valve SV2 allows or disallows the dilution water D in the dilution water tank 20 to be injected into the container 30. Specifically, by opening the solenoid valve SV2 until the second liquid level detection portion 42 detects the second liquid level L2, the dilution water D in the dilution water tank 20 is injected into the container 30 through the pipes 21 and 22. As a result, the raw water R in the container 30 is diluted with the dilution water D, and the sample water S consisting of the raw water R and the dilution water D is prepared. After that, when the second liquid level detection portion 42 detects the second liquid level L2, the injection of the dilution water D into the container 30 is stopped by closing the solenoid valve SV2. The opening or closing of the solenoid valve SV2 may be controlled, for example, by the control device described above.

A predetermined amount of dilution water D may be injected into the container 30 by operating the dilution water injection portion as described above. The amount of dilution water D to be injected is increased or decreased by changing the position of the second liquid level L2 detected by the second liquid level detection portion 42 upward or downward.

In the dilution device 100, predetermined amounts of raw water R and dilution water D are injected into the container 30, and the raw water R is diluted. As a result, the raw water R is diluted to a predetermined ratio, and the sample water S is prepared. The dilution ratio X of the raw water R in the dilution of the raw water R is calculated by the amounts of raw water R, dilution water D, and sample water S as follows. Here, the amount A_{R} of raw water R injected into the container 30 is the amount when the water surface of the raw water R reaches the first liquid level L1. The amount A_{D} of dilution water D injected into the container 30 is the amount injected until the water surface of the sample water S reaches the second liquid level L2. That is, the amount A_{D} of dilution water D injected into the container 30 is also the amount obtained by subtracting the amount A_{R} of raw water R from the amount A_{S} of sample water S when the water surface of the sample water S reaches the second liquid level L2 (A_{D}=A_{S}−A_{R}). The dilution ratio X of the raw water R is calculated by the following equation.

$$Dilution ratio X [ times ] = {A}_{S} / {A}_{R} = ( {A}_{R} + {A}_{D} ) / {A}_{R}$$

In the dilution device 100, after the dilution of the raw water R is performed as described above, a re-dilution of the raw water R may be performed. In the dilution device 100, the raw water R may be re-diluted two or more times.

Here, in this description, “dilution of raw water” specifically means diluting raw water by injecting dilution water into the raw water in a container. “Re-dilution of raw water” specifically means diluting the raw water again by injecting the dilution water into the sample water including the raw water in the container after the dilution of raw water described above. Thus, in the dilution device, in the case where the dilution of raw water is performed once and then the re-dilution of raw water is performed twice, the raw water is diluted a total of three times. Hereinafter, a case where the dilution device performs the re-dilution of raw water will be described.

The dilution device 100 may further include a discharge portion that discharges the sample water S accommodated in the container 30 until the first liquid level detection portion 41 detects the first liquid level L1. The discharge portion may include, for example, a pipe 51, a solenoid valve SV3, pipes 52, 53, 54, a solenoid valve SV6, and a pipe 55 connected to the bottom surface portion 32 of the container 30. The opening or closing of the solenoid valves SV3 and SV6 allows or disallows the sample water S accommodated in the container 30 to be discharged. Specifically, by opening the solenoid valves SV3 and SV6 until the first liquid level detection portion 41 detects the first liquid level L1, the sample water S in the container 30 is discharged as waste water W through the pipes 51, 52, 53, 54, and 55. After that, when the first liquid level detection portion 41 detects the first liquid level L1, the discharge of the sample water S accommodated in the container 30 is stopped by closing the solenoid valves SV3 and SV6. The opening or closing of the solenoid valves SV3 and SV6 may be controlled, for example, by the control device described above.

After the discharge portion discharges the sample water S from the container 30 and the liquid level of the sample water S reaches the first liquid level L1, re-dilution may be performed. That is, in the dilution device 100, after the discharge portion discharges the sample water S, the dilution water injection portion may re-dilute the raw water R by injecting the dilution water D into the container 30 until the second liquid level detection portion 42 detects the second liquid level L2. The injection of the dilution water S in the re-dilution may be performed by opening the solenoid valve SV2 until the second liquid level detection portion 42 detects the second liquid level L2, as described above.

When the re-dilution of raw water R is performed Y times with the same dilution ratio as the dilution ratio X of the raw water R, the final dilution ratio X_{Y} of the raw water R is calculated by the following equation.

$$Dilution ratio {X}_{Y} [ times ] = {X}^{Y + 1}$$

In the dilution device 100, the dilution ratio in the dilution and the re-dilution may be the same or different. That is, the dilution ratio may be changed after the dilution and before the re-dilution of the raw water R. Further, in the case where the re-dilution of raw water R is performed two or more times, the dilution ratio may be changed after the re-dilution and before the next re-dilution. The change in the dilution ratio is performed by changing the position of the first liquid level L1 detected by the first liquid level detection portion 41 and/or the position of the second liquid level L2 detected by the second liquid level detection portion 42. That is, in the dilution device 100, the position of the first liquid level L1 detected by the first liquid level detection portion 41 and/or the position of the second liquid level L2 detected by the second liquid level detection portion 42 may be changed after the dilution and before the re-dilution of the raw water R. Further, in the case where the re-dilution of raw water R is performed two or more times, the position of the first liquid level L1 detected by the first liquid level detection portion 41 and/or the position of the second liquid level L2 detected by the second liquid level detection portion 42 may be changed after the re-dilution and before the next re-dilution.

The dilution ratio in the dilution device 100 and the number of re-dilutions may be set in accordance with the residual chlorine concentration of the raw water R and the residual chlorine concentration measurable by the residual chlorine concentration measurement device 200. In this embodiment, the residual chlorine concentration of the raw water R may exceed the concentration measurable by the residual chlorine concentration measurement device 200. Thus, it is preferable that the residual chlorine concentration of the raw water R is measured in advance by manual analysis, and the dilution ratio and the number of re-dilutions are set so that the concentration is measurable by the residual chlorine concentration measurement device 200.

In the case where the final dilution ratio of the raw water R is high (e.g., several tens of times or more), to reduce errors during dilution, preferably, multiple dilutions (i.e., one or more re-dilutions) are performed. For example, in the case where the final dilution ratio of the raw water R is 1000 times (the raw water R 1000 times diluted), the raw water R may be 1000 times diluted in one dilution, but preferably 1000 times diluted in multiple dilutions. Specifically, for example, the raw water R may be 10 times diluted in the first dilution, then further 10 times diluted by a second dilution (first re-dilution), and further 10 times diluted by a third dilution (second re-dilution). As a result, the final dilution ratio of the raw water R becomes 1000 times.

The dilution device 100 may further include a circulation portion that circulates the sample water S within the container 30. Thereby, the sample water S in the container 30 may be made uniform. By making the sample water S uniform, the measurement accuracy of the residual chlorine concentration by the residual chlorine concentration measurement device 200 may be improved.

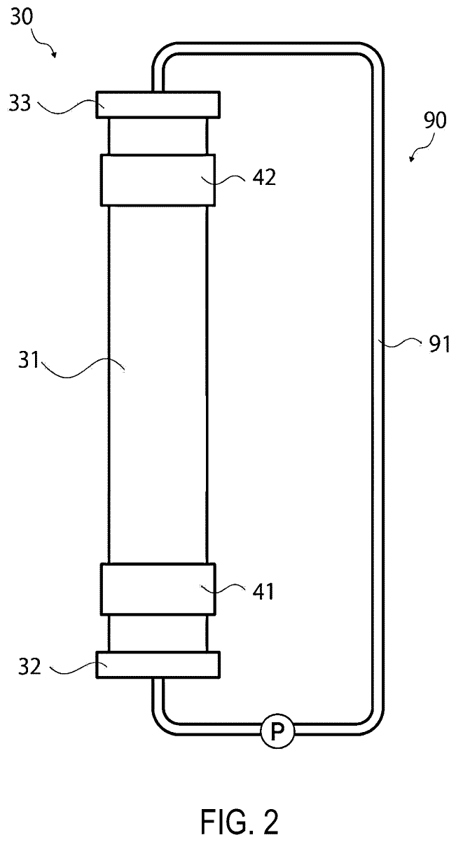

FIG. 2 is a schematic view showing the container 30 and the circulation portion 90. The circulation portion 90 may include, for example, a flow path 91 and a pump P that connect the bottom surface portion 32 and the top surface portion 33. The circulation portion 90 may, for example, inject the sample water S discharged from the bottom surface portion 32 into the container 30 from the top surface portion 33 via the flow path 91. Thereby, the sample water S in the container 30 may be circulated from the bottom to the top. It is noted that the pump P shown in FIG. 2 may be the same as or different from the pump Pin FIG. 1, which will be described later.

Further, the circulation portion 90 may include valves (not shown). The valves may be, for example, the solenoid valves SV3 and SV4 shown in FIG. 1.

In the dilution device 100, the circulation of the sample water S by the circulation portion 90 may be performed once or multiple times. The circulation of the sample water S is preferably performed after the dilution of raw water R and after the re-dilution of raw water R.

The sample water S prepared in the dilution device 100 is preferably transferred to the sample water storage portion 70 and then provided to the residual chlorine concentration measurement device 200.

Next, an example of a treatment process in which the dilution device 100 prepares the sample water S and transfers the same to the sample water storage portion 70 will be described with reference to Table 1 below. In one example of the treatment process, the dilution device 100 is set so that the dilution ratio in one dilution is 10 times. Specifically, in the container 30 of the dilution device 100, it is set so that the water amount when reaching the first liquid level L1 is 10% (10 mL), and the water amount when reaching the second liquid level L2 is 100% (100 mL). Thus, by injecting the raw water R until reaching the first liquid level L1 and injecting the dilution water D until reaching the second liquid level L2, a 10 times diluted sample water S (100 mL) is prepared by mixing 10% (10 mL) of the raw water R and 90% (90 mL) of the dilution water D.

Table 1 below shows the states of the dilution device 100, the solenoid valves SV1 to SV6, and the pump P in an example of the above treatment process. In Table 1, when the pump P is in the “ON” state, the pump is driven and water is transferred.

TABLE 1

| | | Solenoid valve | | | | | | |
|---|---|---|---|---|---|---|---|---|
| Step | Dilution device | SV1 | SV2 | SV3 | SV4 | SV5 | SV6 | Pump P |
| S0 | Initial | Closed | Closed | Closed | Closed | Closed | Closed | Off |
| S1 | Inject 100% raw | Open | Closed | Closed | Closed | Closed | Closed | Off |
| | water started | | | | | | | |
| S2 | Inject 100% raw | Closed | Closed | Closed | Closed | Closed | Closed | Off |
| | water stopped | | | | | | | |
| S3 | Discharge all raw | Closed | Closed | Open | Closed | Closed | Open | Off |
| | water in the | | | | | | | |
| | container started | | | | | | | |
| S4 | Discharge all raw | Closed | Closed | Closed | Closed | Closed | Closed | Off |
| | water in the | | | | | | | |
| | container stopped | | | | | | | |
| S5 | Discharge all raw | Closed | Closed | Open | Open | Closed | Open | ON |
| | water in the | | | | | | | |
| | pipe started | | | | | | | |
| S6 | Discharge all raw | Closed | Closed | Closed | Closed | Closed | Closed | Off |
| | water in the | | | | | | | |
| | pipe stopped | | | | | | | |
| S7 | Inject 10% raw water | Open | Closed | Closed | Closed | Closed | Closed | Off |
| | started | | | | | | | |
| S8 | Inject 10% raw water | Closed | Closed | Closed | Closed | Closed | Closed | Off |
| | stopped | | | | | | | |
| S9 | Inject 90% dilution | Closed | Open | Closed | Closed | Closed | Closed | Off |
| | water started | | | | | | | |
| S10 | Inject 90% dilution | Closed | Closed | Closed | Closed | Closed | Closed | Off |
| | water stopped | | | | | | | |
| S11 | Sample water | Closed | Closed | Open | Open | Closed | Closed | ON |
| | circulation started | | | | | | | |
| S12 | Sample water | Closed | Closed | Closed | Closed | Closed | Closed | Off |
| | circulation stopped | | | | | | | |
| S13 | Discharge the sample | Closed | Closed | Open | Closed | Closed | Open | Off |
| | water from the | | | | | | | |
| | container to 10% | | | | | | | |
| | started | | | | | | | |
| S14 | Discharge the sample | Closed | Closed | Closed | Closed | Closed | Closed | Off |
| | water from the | | | | | | | |
| | container to 10% | | | | | | | |
| | stopped | | | | | | | |
| S15 | Discharge water in | Closed | Closed | Open | Open | Closed | Open | ON |
| | the pipe started | | | | | | | |
| S16 | Discharge water in | Closed | Closed | Closed | Closed | Closed | Closed | Off |
| | the pipe stopped | | | | | | | |
| S17 | Inject 90% dilution | Closed | Open | Closed | Closed | Closed | Closed | Off |
| | water started | | | | | | | |
| S18 | Inject 90% dilution | Closed | Closed | Closed | Closed | Closed | Closed | Off |
| | water stopped | | | | | | | |
| S19 | Sample water | Closed | Closed | Open | Open | Closed | Closed | ON |
| | circulation started | | | | | | | |
| S20 | Sample water | Closed | Closed | Closed | Closed | Closed | Closed | Off |
| | circulation stopped | | | | | | | |
| S21 | Transfer the sample | Closed | Closed | Open | Closed | Open | Closed | Off |
| | water to the sample | | | | | | | |
| | water storage | | | | | | | |
| | portion started | | | | | | | |
| S22 | Transfer the | Closed | Closed | Closed | Closed | Closed | Closed | Off |
| | sample water to | | | | | | | |
| | the sample water | | | | | | | |
| | storage portion | | | | | | | |
| | stopped | | | | | | | |
| S23 | Discharge water in | Closed | Closed | Closed | Open | Closed | Open | Off |
| | the pipe started | | | | | | | |
| S24 | Discharge water in | Closed | Closed | Closed | Closed | Closed | Closed | Off |
| | the pipe stopped | | | | | | | |

In Table 1 above, step S0 is the initial state. In step S0, all solenoid valves SV1 to SV6 are closed and the pump P is off.

In step S1, the solenoid valve SV1 is opened. Thereby, “inject 100% raw water” is started. In the example of Table 1, “inject 100% raw water” means that the raw water R is injected into the container 30 until reaching the second liquid level L2. Specifically, this means that the raw water R is injected into the container 30 until the second liquid level detection portion 42 detects the second liquid level L2. Next, in step S2, the solenoid valve SV1 is closed. Thereby, “inject 100% raw water” is stopped, and the state becomes such that only the raw water R is injected into the container 30 up to the second liquid level L2.

In step S3, the solenoid valves SV3 and SV6 are opened. Thereby, “discharge all raw water in the container” is started. Next, in step S4, the solenoid valves SV3 and SV6 are closed. Thereby, “discharge all raw water in the container” is stopped, and the state becomes such that the container 30 is empty. In steps S3 and S4, the inner wall of the container 30 is washed by the raw water R.

In step S5, the solenoid valves SV3, SV4, and SV6 are opened, and the pump P is turned ON. Thereby, “discharge all raw water in the pipe” is started. Next, in step S6, the solenoid valves SV3, SV4, and SV6 are closed, and the pump P is turned off. Thereby, “discharge all raw water in the pipe” is stopped.

In step S7, the solenoid valve SV1 is opened. Thereby, “inject 10% raw water” is started. In the example of Table 1, “inject 10% raw water” means that the raw water R is injected into the container 30 until reaching the first liquid level L1. Specifically, this means that the raw water R is injected into the container 30 until the first liquid level detection portion 41 detects the first liquid level L1. Next, in step S8, the solenoid valve SV1 is closed. Thereby, “inject 10% raw water” is stopped, and the state becomes such that the raw water R is injected into the container 30 up to the first liquid level L1.

In step S9, the solenoid valve SV2 is opened. Thereby, “inject 90% dilution water” is started. In the example of Table 1, “inject 90% dilution water” means that the dilution water D is injected into the container 30 until reaching the second liquid level L2. Specifically, this means that the dilution water D is injected into the container 30 until the second liquid level detection portion 42 detects the second liquid level L2. Next, in step S10, the solenoid valve SV2 is closed. Thereby, “inject 90% dilution water” is stopped, and the state becomes such that the dilution water D is injected into the container 30 up to the second liquid level L2. As a result, the sample water S including 10% raw water R and 90% dilution water D is prepared in the container 30. That is, the dilution of the raw water R is completed, and the raw water R is 10 times diluted in the container 30.

In step S11, the solenoid valves SV3 and SV4 are opened, and the pump P is turned ON. Thereby, “sample water circulation” is started. That is, in the example of Table 1, the circulation portion 90 shown in FIG. 2 has the solenoid valves SV3, SV4, and the pump P shown in FIG. 1. Next, in step S12, the solenoid valves SV3 and SV4 are closed, and the pump P is turned off. Thereby, “sample water circulation” is stopped. In addition, in the example of Table 1, the “sample water circulation” may be, for example, performed for 30 seconds at a rate of 300 mL/min.

In step S13, the solenoid valves SV3 and SV6 are opened. Thereby, “discharge the sample water from the container to 10%” is started. In the example of Table 1, “discharge the sample water from the container to 10%” means that the sample water S in the container is discharged until reaching the first liquid level L1. Specifically, this means that the sample water S in the container is discharged until the first liquid level detection portion 41 detects the first liquid level L1. Next, in step S14, the solenoid valves SV3 and SV6 are closed. As a result, “discharge the sample water from the container to 10%” is stopped, and the state becomes such that 10% (10 mL) of the sample water S remains in the container 30.

In step S15, the solenoid valves SV3, SV4, and SV6 are opened, and the pump P is turned ON. Thereby, “discharge water in the pipe” is started. Next, in step S16, the solenoid valves SV3, SV4, and SV6 are closed, and the pump P is turned off. Thereby, “discharge water in the pipe” is stopped.

In step S17, the solenoid valve SV2 is opened. Thereby, “inject 90% dilution water” is started. Next, in step S18, the solenoid valve SV2 is closed. Thereby, “inject 90% dilution water” is stopped, and the state becomes such that the dilution water D is injected into the container 30 up to the second liquid level L2. As a result, the re-diluted sample water S (100 mL) including 10% sample water S (10 mL) and 90% dilution water D (90 mL) is prepared in the container 30. That is, the re-dilution of the raw water R is completed, and the raw water R is 100 times diluted in the container 30.

In step S19, the solenoid valves SV3 and SV4 are opened, and the pump P is turned ON. Thereby, “sample water circulation” is started. Next, in step S20, the solenoid valves SV3 and SV4 are closed, and the pump P is turned off. Thereby, “sample water circulation” is stopped.

In step S21, the solenoid valves SV3 and SV5 are opened. Thereby, “transfer the sample water to the sample water storage portion” is started, and the sample water S in the container 30 is transferred to the sample water storage portion 70. Next, in step S22, the solenoid valves SV3 and SV5 are closed. Thereby, “transfer the sample water to the sample water storage portion” is stopped.

In step S23, solenoid the valves SV4 and SV6 are opened. Thereby, “discharge water in the pipe” is started. Next, in step S24, the solenoid valves SV4 and SV6 are closed. Thereby, “discharge water in the pipe” is stopped.

According to an example of the treatment process detailed above, the raw water R may be 100 times diluted, and the prepared sample water S may be transferred to the sample water storage portion 70. It is noted that when the raw water R is to be 1000 times diluted, steps S13 to S20 may be performed again after step S20 in Table 1 above.

### 1-2. Sample Water Storage Portion

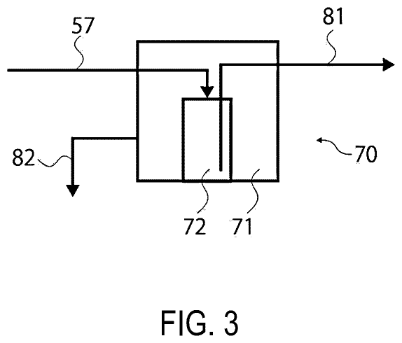

The configuration of the sample water storage portion 70 and the procedure for transferring the sample water from the dilution device 100 to the sample water storage portion 70 will be described with reference to FIG. 1 and FIG. 3. FIG. 3 is a schematic view showing the sample water storage portion 70. As shown in FIG. 1 and FIG. 3, the sample water storage portion 70 includes, for example, a sample water tank 71 and a sample water container 72 configured inside the sample water tank 71.

The dilution device 100 may include a sample water transfer portion that transfers the sample water S in the container 30 to the sample water storage portion 70. The sample water transfer portion may include, for example, a pipe 51, a solenoid valve SV3, pipes 52, 53, and 56, a solenoid valve SV5, and a pipe 57 connected to the bottom surface portion 32 of the container 30, as shown in FIG. 1. The opening or closing of the solenoid valves SV3 and SV5 allows or disallows the sample water S in the container 30 to be transferred to the sample water storage portion 70. Specifically, by opening the solenoid valves SV3 and SV5, the sample water S in the container 30 is transferred to the sample water storage portion 70 through the pipes 51, 52, 53, 56, and 57. As shown in FIG. 1 and FIG. 3, the sample water S that has passed through the pipe 57 may be accommodated in the sample water container 72. The sample water S accommodated in the sample water container 72 may be provided to the residual chlorine concentration measurement device 200 through a pipe 81. On the other hand, the sample water S overflowing from the sample water container 72 and flowing into the sample water tank 71 may be discharged as the waste water W through a pipe 82.

By providing the sample water storage portion 70, the sample water S may be temporarily stored before being transferred to the residual chlorine concentration measurement device 200. As a result, it is easier to adjust the amount and timing of the sample water S to be sent to the residual chlorine concentration measurement device 200.

### 1-3. Residual Chlorine Concentration Measurement Device

The sample water S in the container 30 is finally transferred to the residual chlorine concentration measurement device 200. The residual chlorine concentration measurement device 200 measures the residual chlorine concentration of the sample water S. Preferably, the residual chlorine concentration measurement device 200 is a device that measures the residual chlorine concentration of the sample water S based on an absorbance of the sample water S colored by a reagent. The device may be any device known in the art. Among conventional residual chlorine concentration measurement devices, there is a contact type device in which a measurement sensor performs concentration measurement by directly contacting target water. In that contact type device, scales may deposit to the measurement sensor due to precipitated substances in the target water, causing measurement abnormalities and deterioration over time. On the other hand, in a device that performs concentration measurement using the absorptiometric method, the concentration measurement part does not contact the target water directly, so the above-mentioned measurement abnormalities and deterioration over time are unlikely to occur. Thus, the residual chlorine concentration measurement device using the absorptiometric method is suitable for continuous operation and contributes to reducing the burden of maintenance work.

Examples of the above-mentioned reagent include N,N-diethyl-1,4-phenylenediamine reagent (DPD reagent) and 3,3′-dimethylbenzidine reagent (o-tolidine reagent). The reagent used in the residual chlorine concentration measurement device 200 is preferably the DPD reagent.

The residual chlorine concentration of the raw water R is calculated based on the residual chlorine concentration of the sample water S measured by the residual chlorine concentration measurement device 200 and the dilution ratio of the raw water R in the dilution device 100 (the final dilution ratio if diluted multiple times). The calculation of the residual chlorine concentration of the raw water R may be performed by the residual chlorine concentration measurement device 200, or may be performed by other calculation device (not shown). As a result, the quantification of the residual chlorine concentration of the raw water R is completed.

In the case where the residual chlorine concentration of the sample water S is outside the concentration range measurable by the residual chlorine concentration measurement device 200 (e.g., the case where the residual chlorine concentration of the sample water S is too low or too high), the dilution ratio of the raw water R in the dilution device 100 may be changed in accordance with the residual chlorine concentration of the sample water S. The change of the dilution ratio may be performed by, for example, the control device (not shown). For example, in the case where the residual chlorine concentration of the sample water S is lower than the above-mentioned measurable concentration range, the dilution ratio of the raw water R in the dilution device 100 may be set low, and the residual chlorine concentration of the sample water S may be set to be higher. For example, in the case where the residual chlorine concentration of the sample water S is higher than the above-mentioned measurable range, the dilution ratio of the raw water R in the dilution device may be set high, and the residual chlorine concentration of the sample water S may be set to be lower.

### 2. Effect of Water Quality Measurement Device

As detailed above, in the water quality measurement device of this embodiment, the raw water is diluted by a dilution device before being provided to the residual chlorine concentration measurement device. The sample water prepared by diluting the raw water becomes the object of measurement of the residual chlorine concentration measurement device. Thus, even when the residual chlorine concentration of the raw water exceeds the concentration measurable by the residual chlorine concentration measurement device, the residual chlorine concentration of the raw water may be quantified by measuring the residual chlorine concentration of the sample water. That is, the water quality measurement device of this embodiment may automatically quantify high-concentration residual chlorine concentration. By using the water quality measurement device of this embodiment, the residual chlorine concentration may be automatically and continuously quantified in a water system with a high residual chlorine concentration.

Further, generally, in the case where the pH of the raw water fluctuates over time, the residual chlorine concentration of the raw water continuously measured by the device may be affected by the pH fluctuations. On the other hand, in the water quality measurement device of this embodiment, since the raw water provided to the residual chlorine concentration measurement device is in a diluted state, the influence of the pH fluctuations of the raw water on the residual chlorine concentration measurement may be reduced. Furthermore, even when there are factors in the raw water that may interfere with the measurement of residual chlorine concentration (e.g., when the raw water is colored, furthermore, even when the raw water is contaminated with chlorine ions, salts, etc.), the influence of these factors on the measurement of residual chlorine concentration may be reduced by diluting the raw water. As a result, the accuracy of the residual chlorine concentration measurement may be improved.

Further, generally, when the pH of the raw water is high (e.g., pH 9 or higher), there is a concern about the deterioration over time of the measurement device and pipe due to alkali corrosion. On the other hand, in the water quality measurement device of this embodiment, deterioration of the measurement device, pipe, etc. may be suppressed due to the pH buffering effect due to the dilution of raw water.

### 3. Usage of Water Quality Measurement Device

As described above, the water quality measurement device of this embodiment is suitable for quantifying the residual chlorine concentration of raw water with a high residual chlorine concentration. For this reason, the water quality measurement device may be used, for example, in cleaning in place (CIP), where washing water containing a high concentration of residual chlorine is used. That is, the water quality measurement device of this embodiment may be for cleaning in place, and the raw water used in the water quality measurement device may be washing water for cleaning in place.

In addition, the water quality measurement device of this embodiment is suitable for use in, for example, water systems where the pH may fluctuate greatly and water systems (e.g., ballast water) mixed with factors interfering with residual chlorine concentration measurements (e.g., chlorine ions and salts). In addition, the water quality measurement device may be used, for example, in water systems with long residence times in the storage pits of air conditioning equipment. Cleaning agents, disinfectants, corrosion inhibitors, etc. are thrown into the storage pits, and in the case where dirt due to slime or scale or acid/alkaline agents is present, the residual chlorine concentration may vary depending on the reaction between the water system and the agent. In such a water system, the water quality measurement device of this embodiment may be applied.

### EXAMPLES

The present invention will be described in more detail below with reference to the Example, but the present invention is not limited to the Example.

### Test Example 1

A test was conducted to automatically measure residual chlorine concentration in raw water using the water quality measurement device shown in FIG. 1 to FIG. 3. The residual chlorine concentration measurement device included in the water quality measurement device was a device that measures the residual chlorine concentration by the absorptiometric method using a color reaction caused by a DPD reagent. The device was provided with a measurement cell that accommodates sample water to be measured, and in the device, the measurement cell for residual chlorine concentration of 0.00 to 2.00 mg/L and the measurement cell for residual chlorine concentration of 0.00 to 8.00 mg/L may be replaced as necessary. In this test, the sample water was prepared by 100 times diluting the raw water with residual chlorine concentration of 500 mg/L in the dilution device, and the residual chlorine concentration of the sample water was automatically measured using the measurement cell for 0.00 to 8.00 mg/L in the residual chlorine concentration measurement device.

Moreover, apart from the measurement using the water quality measurement device described above, measurement was also performed by manual analysis. Specifically, the tester manually collected the raw water and the sample water accommodated in the sample water storage portion, respectively, and the residual chlorine concentrations of both were measured by the above-mentioned residual chlorine concentration measurement device using the measurement cell for 0.00 to 2.00 mg/L. The procedure for concentration measurement is described below.

First, the residual chlorine concentration of raw water in the raw water tank was measured by manual analysis. Next, the above-mentioned water quality measurement device was operated, and after the start of the operation, automatic measurements using the residual chlorine concentration measurement device were performed at time points of 9 minutes, 12 minutes, 19 minutes, 22 minutes, 25 minutes, 28 minutes, 32 minutes, 35 minutes, 38 minutes, 41 minutes, 44 minutes, and 47 minutes. In addition, after the start of the above-mentioned operation, the raw water was collected at time points of 19 minutes and 44 minutes, the sample water in the sample water storage portion was collected at time points of 9 minutes, 25 minutes, and 44 minutes, and the residual chlorine concentrations of the raw water and the sample water were measured by manual analysis.

The results of the concentration measurement in Test Example 1 are shown in Table 2 below.

TABLE 2

| | Residual | | |
|---|---|---|---|
| | chlorine | | |
| | concentration | | |
| | of raw water | Residual chlorine concentration of | |
| | [mg/L] | sample water [mg/L] | |
| Time | Manual | Residual chlorine concentration | Manual |
| (Minutes) | analysis | measurement device | analysis |
| 0 | 0.48 | — | — |
| 9 | — | 4.79 | 0.47 |
| 12 | — | 4.68 | — |
| 19 | 0.47 | 4.25 | — |
| 22 | — | 4.25 | — |
| 25 | — | 4.59 | 0.46 |
| 28 | — | 4.63 | — |
| 32 | — | 4.66 | — |
| 35 | — | 4.71 | — |
| 38 | — | 4.68 | — |
| 41 | — | 4.65 | — |
| 44 | 0.47 | 4.70 | 0.46 |
| 47 | — | 4.70 | — |

### Test Example 2

Test Example 2 was conducted using the same procedure as Test Example 1, except that the residual chlorine concentration in the raw water was set to 700 mg/L, the dilution ratio of the raw water was set to 1000 times, and the measurement cell for 0.00 to 2.00 mg/L was used in the automatic measurement, and after the water quality measurement device started operating, automatic measurements were performed at time points of 9 minutes, 12 minutes, 30 minutes, 36 minutes, and 40 minutes, while manual analysis of the raw water and the sample water were performed at time points of 9 minutes and 40 minutes. The results of the concentration measurement in Test Example 2 are shown in Table 3 below.

TABLE 3

| | Residual chlorine | | |
|---|---|---|---|
| | concentration | Residual chlorine concentration | |
| | of raw water | of sample water [mg/L] | |
| Time | [mg/L] | Residual chlorine concentration | Manual |
| (Minutes) | Manual analysis | measurement device | analysis |
| 0 | 0.74 | — | — |
| 9 | 0.74 | 0.76 | 0.75 |
| 12 | — | 0.75 | — |
| 30 | — | 0.76 | — |
| 36 | — | 0.75 | — |
| 40 | 0.74 | 0.74 | 0.75 |

As shown in Tables 2 and 3 above, the concentration of the sample water measured by the residual chlorine concentration measurement device is almost equivalent to the concentration measured by manual analysis. From this, it can be seen that the residual chlorine concentration measured by the water quality measurement device of the present invention is equivalent to the concentration measured by conventional manual analysis.

The present invention may also be in the following aspect.

[1]

A water quality measurement device, comprising:
a dilution device, preparing sample water including raw water and dilution water; and
a residual chlorine concentration measurement device, measuring a residual chlorine concentration of the sample water,
wherein the dilution device comprises:
a container in which the sample water is accommodated;
a first liquid level detection portion, detecting a first liquid level in the container;
a second liquid level detection portion, detecting a second liquid level in the container;
a raw water injection portion, injecting the raw water into the container until the first liquid level detection portion detects the first liquid level; and
a dilution water injection portion, diluting the raw water by injecting the dilution water into the container until the second liquid level detection portion detects the second liquid level.
   [2]

The water quality measurement device according to [1], wherein
the dilution device comprises:
a discharge portion, discharging the sample water accommodated in the container until the first liquid level detection portion detects the first liquid level, and
after the discharge portion discharges the sample water, the dilution water injection portion re-dilutes the raw water by injecting the dilution water into the container until the second liquid level detection portion detects the second liquid level.
   [3]

The water quality measurement device according to [2], wherein the raw water is re-diluted for two or more times in the dilution device.

[4]

The water quality measurement device according to any one of [1] to [3], wherein the dilution device comprises a circulation portion that circulates the sample water within the container.

[5]

The water quality measurement device according to any one of [1] to [4], wherein the residual chlorine concentration measurement device measures the residual chlorine concentration of the sample water based on an absorbance of the sample water colored by a reagent.

[6]

The water quality measurement device according to [5], wherein the reagent is an N,N-diethyl-1,4-phenylenediamine reagent.

[7]

The water quality measurement device according to any one of [1] to [6], wherein the residual chlorine concentration of the raw water is more than 8 mg/L.

[8]

The water quality measurement device according to any one of [1] to [7], wherein the raw water is washing water for cleaning in place.

To the claims:

1. A water quality measurement device, comprising: a dilution device, preparing sample water including raw water and dilution water; and
a residual chlorine concentration measurement device, measuring a residual chlorine concentration of the sample water,
wherein the dilution device comprises:
a container in which the sample water is accommodated;
a first liquid level detection portion, detecting a first liquid level in the container;
a second liquid level detection portion, detecting a second liquid level in the container;
a raw water injection portion, injecting the raw water into the container until the first liquid level detection portion detects the first liquid level; and
a dilution water injection portion, diluting the raw water by injecting the dilution water into the container until the second liquid level detection portion detects the second liquid level.

2. The water quality measurement device according to claim 1, wherein
the dilution device comprises:
a discharge portion, discharging the sample water accommodated in the container until the first liquid level detection portion detects the first liquid level, and
after the discharge portion discharges the sample water, the dilution water injection portion re-dilutes the raw water by injecting the dilution water into the container until the second liquid level detection portion detects the second liquid level.

3. The water quality measurement device according to claim 2, wherein the raw water is re-diluted for two or more times in the dilution device.

4. The water quality measurement device according to claim 1, wherein the dilution device comprises a circulation portion that circulates the sample water within the container.

5. The water quality measurement device according to claim 1, wherein the residual chlorine concentration measurement device measures the residual chlorine concentration of the sample water based on an absorbance of the sample water colored by a reagent.

6. The water quality measurement device according to claim 5, wherein the reagent is an N,N-diethyl-1,4-phenylenediamine reagent.

7. The water quality measurement device according to claim 1, wherein the residual chlorine concentration of the raw water is more than 8 mg/L.

8. The water quality measurement device according to claim 1, wherein the raw water is washing water for cleaning in place.
